(19) United States
(12) Patent Application Publication
(10) Pub. No.: US 2004/0073211 A1
(43) Pub. Date: Apr. 15, 2004

(54) Orthopaedic fixation method and device with delivery and presentation features

(76) Inventors: Ed Austin, Bartlett, TN (US); John Schneider, Memphis, TN (US); Michael W. Mullaney, Kinnelon, NJ (US)

Correspondence Address: CHIEF PATENT COUNSEL, SMITH & NEPHEW, INC., 1450 BROOKS ROAD, MEMPHIS, TN, 38116, US

(21) Appl. No.: 10/406,977

(22) Filed: Apr. 4, 2003

Related U.S. Application Data
(60) Provisional application No. 60/370,201, filed on Apr. 5, 2002.

(51) Int. Cl. A61B 17/58

(52) U.S. Cl. 606/54

(57) ABSTRACT

Embodiments of the present invention include devices and methods for aligning fragments of a fractured bone or for positioning bones. In some embodiments, fixation devices and anatomical features are modeled with the aid of a computer served over a network, and the model is used to determine how an actual fixation device should be configured to align or position the bones.

### RELATED APPLICATION DATA

This application claims the benefit of U.S. S No. 60/370,201, filed Apr. 5, 2002 entitled “Orthopaedic Fixation Method and Device” which is incorporated herein by this reference.

### TECHNICAL FIELD

Embodiments of the invention are directed to treating musculoskeletal conditions, including skeletal fractures. More specifically, apparatuses and methods for securing and placing fragments of a fractured bone or bones on two sides of a joint in desired locations are disclosed. In some embodiments of the invention, apparatuses and methods are used to generate a computer model of a fixation device and bones or bone fragments. Through operations on the model, desired placement of the bones or bone fragments is determined quickly and accurately regardless of the initial configuration of the fixation device. The operations required to create the desired placement of the bones or bone fragments may then be enacted on a corresponding physical device to treat the musculoskeletal condition.

### BACKGROUND OF THE INVENTION

Devices and methods of treating skeletal fractures using ring external fixation structures are well known in the art. Smith & Nephew, Inc. has developed and marketed a number of SPATIAL FRAMES brand external ring fixators based on the general concept of a Stewart platform. Smith & Nephew, Inc. owns U.S. Pat. Nos. 5,702,389; 5,728,095; 5,891,143; 5,971,984; 6,030,386; and 6,129,727 that disclose many basic concepts of and improvements to Stewart platform based external fixators. The disclosure of those patents is incorporated by reference herein.

As will be appreciated by one skilled in the art, mathematically solving for the relative positions of the members of a Stewart platform creates a somewhat cumbersome equation. As an example, note the rotational matrix detailed in U.S. Pat. No. 5,971,984. This rotational matrix is a means by which one Stewart platform fixation element can be transformed relative to another to align fragments of a bone with inputs commonly obtainable from a clinical examination. However, in order to use the rotational matrix, a starting position for the fixation elements must be known. Therefore, prior art systems typically have required a Stewart platform type ring fixator to either start or end its transformation in a neutral position. A neutral position is a position where all of the six struts are the same length, and consequently, the rings of the fixator are parallel to one another. See FIGS. 4 and 5. A neutral position makes locating the starting positions of the fixation elements readily calculable. Once the frame moves beyond neutral, Cartesian coordinates of the frame components are difficult to find mathematically. This limitation results in complexity with regard to a mathematical solution for a Stewart platform. As a practical matter, it means that in the past, supposed correction solutions that did not in fact solve a particular deformity were very difficult to secondarily correct. This situation will be described in more detail below as a “crooked-frame/crooked-bone” situation. A solution for the crooked-frame/crooked-bone situation will be described as a “total residual” solution.

The current SPATIAL FRAME® brand external fixators include operating modes for “chronic” and “residual” corrections. A chronic correction is a correction that starts with a fixator frame that has been deformed to fit onto a deformed bone structure such that when the fixator is returned to a given neutral position, the deformed structure will be corrected. In other words, a chronic correction starts with a frame that has been deformed identically to the deformity of the bone.

For a residual correction, a neutral fixator frame is fit onto a deformed structure, and the struts of the fixator are adjusted until the deformity is corrected. Therefore, in the case of a residual correction, a straight-frame/crooked-bone is corrected to a crooked-frame/straight-bone. For a chronic correction, a crooked-frame/crooked-bone situation is corrected to a straight-frame/straight-bone. Note that a “total residual” correction differs from a “residual” correction in that a residual must start with a neutral frame. A total residual may start with even a crooked frame.

The crooked-frame/crooked-bone complication exists where, at the end of a correction, both the bone structure and the frame are crooked. In other words, the deformities of the frame and the bone are different from one another. The current, known mathematical equations are only valid for going to or starting from a neutral frame. Therefore, if a crooked frame is on a crooked bone structure that is not corrected by returning the frame to a neutral position, the current equations will not solve the problem in a single step. Specifically, some of the initial values to plug into the equations cannot be determined. This crooked-frame/crooked-bone situation may result from inaccurate placement or adjustment of a frame, inaccurate x-rays or reading of x-rays used to generate deformity parameters, or any number of inaccurate applications of a device. Such inaccuracies are common and expected, especially in an environment such as a trauma operating room. In the case of a crooked-frame/crooked-bone situation, the surgeon could reset the frame back to neutral and take new x-rays that could be used to establish a new residual correction. However, that would not be optimal for the patient, especially where adjustment of the frame to neutral would result in increased skeletal deformity and pain.

Some crooked-frame/crooked-bone situations may also be solved with a deformity simulator such as the one shown in French Pat. No. 2,576,774, FIG. 6. As shown, two rods that represent segments of bone are connected by hinges about two axes. By setting the rods relative to one another the way the bone segments are actually deformed, noting the position of the simulator, re-aligning the rods, and noting the changes in the simulator, corrective settings for an actual device may be derived. However, this simulator device fails to account for translations or for rotation about all three possible axes between the segments. Both modes of deformation commonly occur. Additionally, manipulation of the mechanical device in the loosened frame would be awkward and potentially require multiple operators.

A total residual solution is highly advantageous over solutions that require more precise alignment of components of the frame with the patient's anatomy. External fixation devices are often used in trauma situations where reduced initial operating time is beneficial to the patient. Total residual devices require relatively little time for alignment and can be x-rayed or imaged and adjusted after the patient has been stabilized. Therefore, an improved device must provide methods and apparatuses for solving crooked-frame/crooked-bone situations.

What is needed are methods and apparatuses that are useful in quickly and accurately determining the strut settings that solve crooked-frame/crooked-bone situations. Optimally, solutions would be obtainable without substitution or experimentation, and all possible physical relationships of bone segments could be modeled. Improved methods and apparatuses may also give a user visual representations of frame placement and correction results so that the parameters the user is inputting are visually verifiable as correct prior to adjustment of the frame on the patient. Visualization also would enable a user to see if pins and wires used in a frame will interfere with strut positions as a correction is executed. Improved methods and apparatuses may be implemented through software that is operative to be run, updated, and replaced over a network either by storage and use on distributed computers or a central computer or a combination of both.

### SUMMARY OF THE INVENTION

An embodiment of the invention is an external orthopaedic fixation device in combination with a computer. In this embodiment, the combination is for aligning fragments of a fractured bone. The orthopaedic fixation device includes a first fixation element for coupling to a first bone fragment and a second fixation element for coupling to a second bone fragment. The device also includes six adjustable length struts coupled at their respective first ends to the first fixation element and coupled at their respective second ends to the second fixation element. When the first bone fragment and the second bone fragment are out of alignment, at least two of the first, second, third, fourth, fifth, and sixth adjustable length struts are different lengths. And in the same embodiment, if the first, second, third, fourth, fifth, and sixth adjustable length struts were the same length, the first bone fragment and the second bone fragment would be out of alignment. The combination is operable to bring the first bone fragment into alignment with the second bone fragment by: storing the relative locations of the first fixation element and the first bone fragment, storing the locations of the couplings of the first ends of the first, second, third, fourth, fifth, and sixth adjustable length struts relative to the first fixation element, storing the relative locations of the second fixation element and the second bone fragment, storing the locations of the couplings of the second ends of the first, second, third, fourth, fifth, and sixth adjustable length struts relative to the second fixation element, spatially associating the stored location of the first fixation element with the stored location of the second fixation element, aligning a computer generated representation of the stored location of the first bone fragment relative to a computer generated representation of the stored location of the second bone fragment, obtaining the respective distances in the aligned computer generated representations between the first and second ends of the first, second, third, fourth, fifth, and sixth adjustable length struts respectively, and providing the aligned lengths of the first, second, third, fourth, fifth, and sixth adjustable length struts to a user for adjusting the adjustable length struts of the external orthopaedic fixation device.

Another embodiment of the invention is a method of configuring an orthopaedic fixation device that can be coupled to fragments of a fractured bone. The method of the embodiment includes representing a first fixation element of the fixation device virtually in three-dimensional space, representing a first bone fragment virtually in three-dimensional space, and spatially associating the representation of the first fixation element with the representation of the first bone fragment. The method also includes representing a second fixation element of the fixation device virtually in three-dimensional space, representing a second bone fragment virtually in three-dimensional space, and spatially associating the representation of the second fixation element with the representation of the second bone fragment. The representation of the first bone fragment is also spatially associated with the representation of the second bone fragment. The method then includes aligning the virtual representation of the first bone fragment with the virtual representation of the second bone fragment while tracking the spatially associated locations of the representation of first fixation element and the representation of the second fixation element, and configuring the orthopaedic fixation device such that the first fixation element is in the same relative position to the second fixation element as the aligned representation of the first fixation element is with the aligned representation of the second fixation element.

Still another embodiment is a method of determining adjustments required to align fragments of a fractured bone coupled in an orthopaedic fixation device that has a first fixation element coupled to a second fixation element by at least three struts, each strut coupled at its first end to the first fixation element and at its second end to the second fixation element. The method in this embodiment includes representing the first fixation element and a first bone fragment in a computer, and spatially associating the representations of the first fixation element with the first bone fragment. The method also includes representing the second fixation element and a second bone fragment in the computer, and spatially associating the representation of the second fixation element with the representation of the second bone fragment. Further, the method includes spatially associating the representation of the first bone fragment with the representation of the second bone fragment, and aligning the representation of the first bone fragment with the representation of the second bone fragment. The location of the representation of the first fixation element relative to the representation of the second fixation element subsequent to the aligning of the representation of the first bone fragment and the representation of the second bone fragment is determined, and the distance between the couplings of each of the at least three struts to the representation of the first fixation element and the representation of the second fixation element is determined. The amount to adjust each of the at least three struts to equal the determined distance between couplings may then be determined.

Yet another embodiment of the invention is a digital computing device programmed to provide data to a user for adjusting an orthopaedic fixation device that can be coupled to fragments of a fractured bone. The digital computing device may include a motherboard, a central processing unit electrically coupled to the motherboard for executing program instructions, a monitor electrically coupled to the motherboard for displaying representations of the fixation device, and a memory device electrically coupled to the motherboard. The memory device stores program instructions that enable the computing device to represent a first fixation element of the fixation device virtually in three-dimensional space, represent a first bone fragment virtually in three-dimensional space, and spatially associate the virtual representation of the first fixation element with the virtual representation of the first bone fragment. Stored instructions also enable the computing device to represent a second fixation element of the fixation device virtually in three-dimensional space, represent a second bone fragment virtually in three-dimensional space, and spatially associate the virtual representation of the second fixation element with the virtual representation of the second bone fragment. The program instructions also enable the computing device to spatially associate the virtual representation of the first bone fragment with the virtual representation of the second bone fragment, align the virtual representation of the first bone fragment with the virtual representation of the second bone fragment while tracking the spatially associated locations of the virtual representation of first fixation element and the virtual representation of the second fixation element, and output data specifying how the first fixation element is to be positioned relative to the second fixation element to align the first bone fragment and the second bone fragment

Another embodiment of the invention is a program storage device containing instructions that enable a computer to provide data specifying how to configure an orthopaedic fixation device that can be coupled to fragments of a fractured bone. Execution of the instructions results in providing data specifying how to configure the orthopaedic fixation device such that a first fixation element is in the same relative position to a second fixation element as a virtual representation of the first fixation element is with an aligned, virtual representation of the second fixation element after virtual representations of the bone fragments have been aligned.

An embodiment of the invention is a method of configuring an orthopaedic fixation device that can be coupled to bones on either side of a joint to move the bones relative to one another. Representations of a first fixation element and a first bone are represented virtually in three-dimensional space and spatially associated. Representations of a second fixation element and a second bone are represented virtually in three-dimensional space and spatially associated. The representation of the first bone is associated with the representation of the second bone and the representations are positioned while tracking the spatially associated locations of the representation of first fixation element and the representation of the second fixation element. The orthopaedic fixation device is configured such that the first fixation element is in the same relative position to the second fixation element as the positioned representation of the first fixation element is with the positioned representation of the second fixation element.

### BRIEF DESCRIPTION OF THE DRAWINGS

FIG. 1 is a perspective view of an orthopaedic fixation device.

FIG. 2 is a perspective view of an orthopaedic fixation device coupled to a tibia.

FIG. 3 is a system diagram of an orthopaedic fixation device in combination with a computer.

FIG. 4 is a perspective view of a virtual representation of an orthopaedic fixation device.

FIG. 5 is a perspective view of a virtual representation of an orthopaedic fixation device.

FIG. 6 is an elevation view of a virtual representation of an orthopaedic fixation device.

FIG. 7 is an elevation view of a virtual representation of an orthopaedic fixation device.

FIG. 8 is a perspective view of a virtual representation of an orthopaedic fixation device with some elements removed for clarity.

FIG. 9 is a screen shot illustrating an embodiment of the invention being executed on a Web browser where a user may login to the program.

FIG. 10 is a screen shot illustrating an embodiment of the invention being executed on a Web browser where case information has been input by a user.

FIG. 11 is a screen shot illustrating an embodiment of the invention being executed on a Web browser where deformity definitions are to be input by a user.

FIG. 12 is a screen shot illustrating an embodiment of the invention being executed on a Web browser where deformity definitions have been input by a user.

FIG. 13 is a screen shot illustrating an embodiment of the invention being executed on a Web browser where fixation device parameters have been input by a user.

FIG. 14 is a screen shot illustrating an embodiment of the invention being executed on a Web browser where fixation device mounting parameters have been input by a user.

FIG. 15 is a screen shot illustrating an embodiment of the invention being executed on a Web browser where initial frame strut lengths have been input by a user.

FIG. 16 is a screen shot illustrating an embodiment of the invention being executed on a Web browser displaying an enlarged initial frame AP view.

FIG. 17 is a screen shot illustrating an embodiment of the invention being executed on a Web browser displaying final frame strut lengths and configurations.

FIG. 18 is a screen shot illustrating an embodiment of the invention being executed on a Web browser displaying an enlarged final frame lateral view.

FIG. 19 is a screen shot illustrating an embodiment of the invention being executed on a Web browser displaying a prescription for an alignment.

FIG. 20 is a screen shot illustrating an embodiment of the invention being executed on a Web browser displaying an enlargement of virtual representations of the fixation device, struts, and bone fragments at a point during the prescription for an alignment.

FIG. 21 is a screen shot illustrating an embodiment of the invention being executed on a Web browser displaying a prescription for an alignment.

FIG. 22 is a perspective view of a virtual representation of a fixation device with a virtual representation of a bone fragment.

FIG. 23 is a perspective view of a virtual representation of a fixation device with a virtual representation of a bone fragment, with some elements of the fixation device removed for clarity.

FIG. 24 is a perspective view of a virtual representation of a fixation device with virtual representation of two bone fragments, with some elements of the fixation device removed for clarity.

FIG. 25 is a perspective view such as FIG. 24 with additional elements of the fixation device removed for clarity and to show association.

FIG. 26 is a perspective view of a virtual representation of a fixation device with virtual representations of two bone fragments.

FIG. 27 is a perspective view of a virtual representation of a fixation device in a neutral position with virtual representations of two bone fragments.

FIG. 28 is a perspective view of a virtual representation of a fixation device with virtual representations of two bone fragments that have been aligned.

FIG. 29 is a system diagram of a digital computing device.

### DETAILED DESCRIPTION OF THE INVENTION

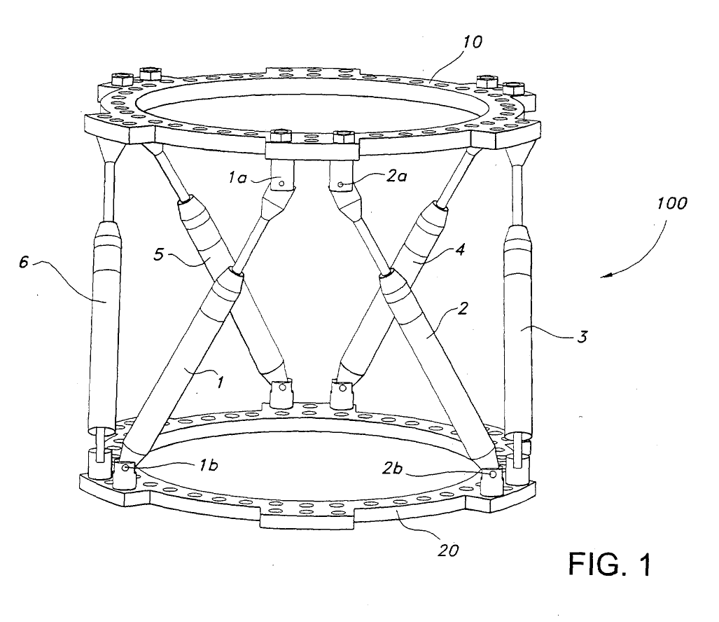

FIG. 1 shows an external orthopaedic fixation device 100 useful for aligning fragments of a fractured bone. The device shown is a Stewart platform based ring fixator device. Smith & Nephew, Inc. markets the particular device shown as a SPATIAL FRAME® brand or TAYLOR SPATIAL FRAME® brand external fixator. The fixation device 100 includes a proximal ring or first fixation element 10 and a distal ring or second fixation element 20. In other embodiments of the invention, the proximal ring could be the second fixation element and the distal ring could be the first fixation element. In FIG. 1, the first fixation element 10 is coupled to the second fixation element 20 by six adjustable length struts 1-6. Each of the struts 1-6 is coupled at its first end to the first fixation element 10 and at its second end to the second fixation element 20.

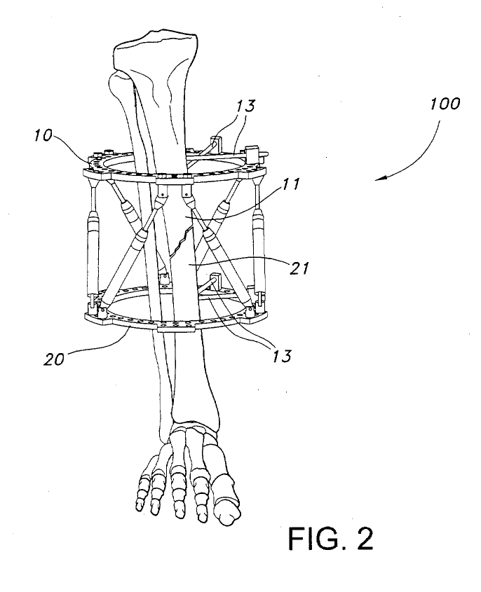

FIG. 2 illustrates the first fixation element 10 coupled to a first bone fragment 11, and the second fixation element 20 coupled to a second bone fragment 21. As shown, the bone fragments are coupled to the fixation elements using cantilevered bone pins 13. In other embodiments, wires, bilateral pins, or any variety of coupling devices effective to secure a bone relative to a fixation element may be used. The fixation device 100 shown is coupled to a tibia; however, the device may be used on practically any bone on which it could be placed. For example, and without limitation, the device could also be used on a femur or a humerus.

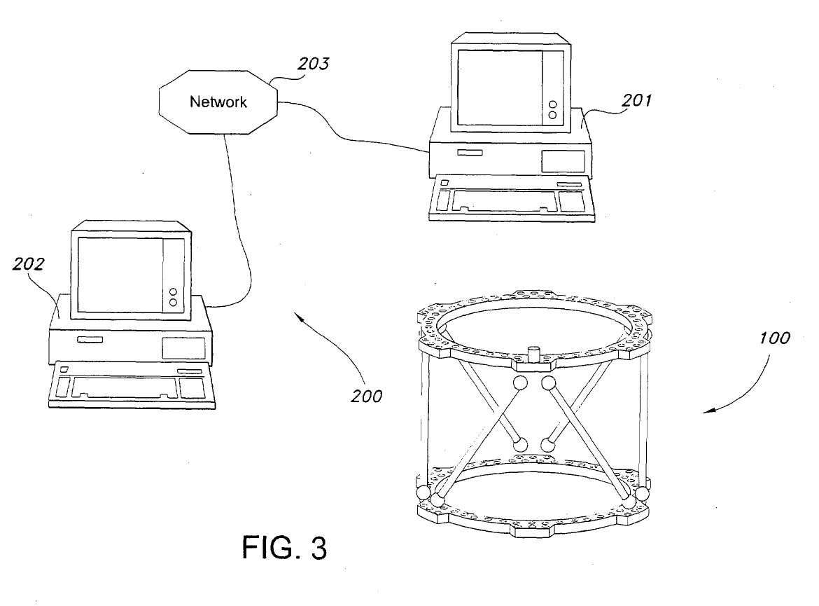

FIG. 3 shows the fixation device 100 in combination with a computer 200. Such a combination is useful to align fragments of a fractured bone. The computer 200 may be an autonomously operating computer system such as, for example, first computer system 201. All storage, processing, etc. necessary to align fragments of a fractured bone may be accomplished with the first computer system 201. In other embodiments, two or more computers may be linked together over a network to accomplish tasks necessary to align the fragments. As shown, computer systems 201 and 202 are linked over a network 203. The network may be a local area network or a wide area network such as the Internet. In some embodiments, all of the programs that are run to accomplish the tasks may be run on one or more of the computer systems, and another of the computer systems may merely be used to display data. Alternatively, the programs run may be run partially on several computer systems, with data and instructions being shared over the network.

For example, in some embodiments, first computer system 201 runs a World Wide Web browser that executes instructions and shares data through network 203 with a second computer system 202 that is a server. This is advantageous in circumstances where a larger computer system is required to run a more complex or memory intensive program. A computer assisted engineering program is an example of such a program. In some embodiments of the present invention, a server computer is used to run both a computer assisted engineering program and to serve or host a World Wide Web site. The term computer assisted engineering program includes both traditional computer aided drafting (CAD) programs, and programs that are capable of not only drafting, but providing design solutions and other data useful in implementing a project. For example, load capacities and dynamic relationships of the components of a structure are provided with some such programs. One computer assisted engineering program useful in the present invention is the Unigraphics program provided by EDS Corporation. Computer assisted engineering and Web hosting functions may themselves be dedicated to separate machines in some embodiments. A served program arrangement may also be beneficial because the supporting programs in such a configuration may be updated by merely updating the program at the central computer or computers. Therefore, software updates become much less complicated and much less expensive.

As described in detail above, a particularly complex situation solved by the present invention is a crooked-frame/crooked-bone situation. Another way of describing the crooked-frame/crooked-bone situation is to say that when two bone fragments are coupled in a fixation device and the fragments are out of alignment, and at least two of the first, second, third, fourth, fifth, and sixth adjustable length struts are different lengths, and if the struts were adjusted until they were any same length, the bone fragments would still be out of alignment. Stated another way, a crooked-frame/crooked-bone situation occurs when both the frame and the attached bone are not neutral or aligned, and the bone would not be aligned if the frame were brought to any neutral position.

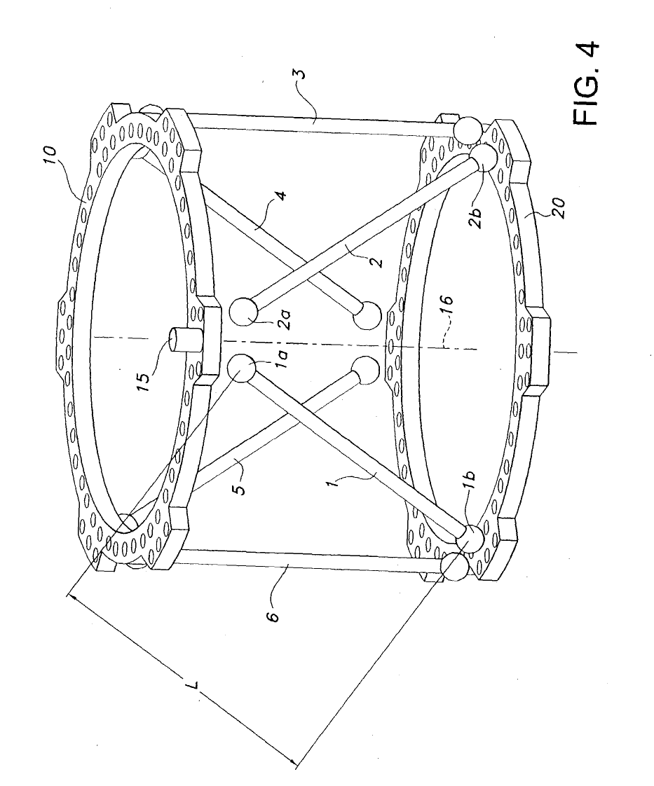

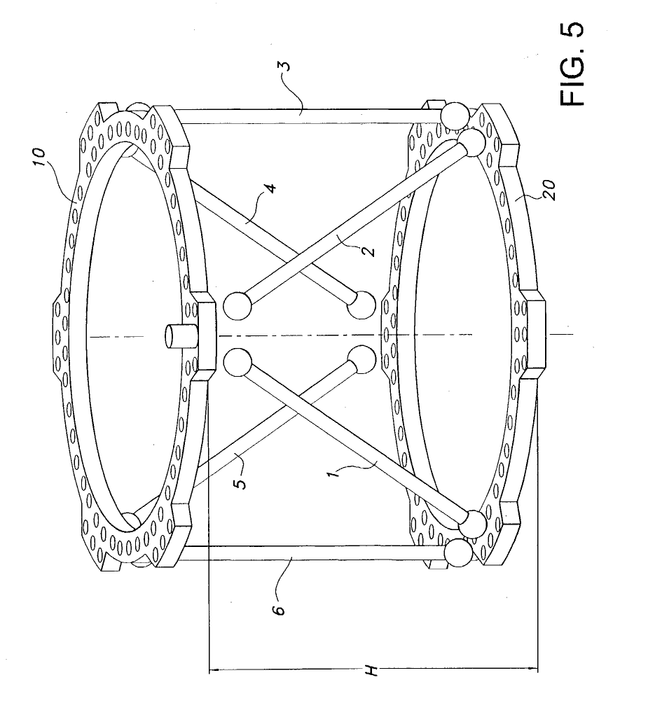

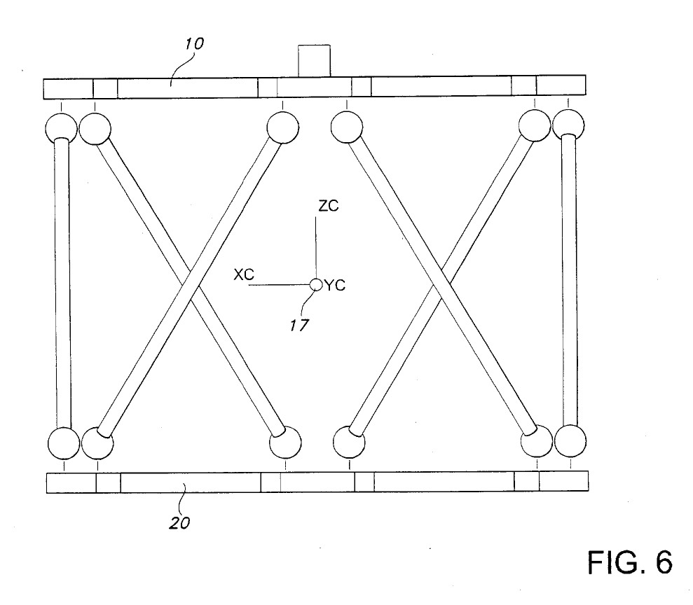

FIGS. 4-8 show geometric characteristics of the fixation device 100. What is shown in the figures are virtual representations of the device generated with the aid of a computer assisted engineering program or the like. A more detailed description of these characteristics will facilitate the discussion of applications of the device that follows. In defining any spatial system, arbitrary points of reference must be established from which to reference the location of components of the system. As illustrated in FIG. 4, the hole between the holes where the proximal ends of struts 1 and 2 are coupled is referred to as a master tab 15. The master tab 15 defines a point of origin in the plane of the first fixation element 10. This point is extended distally and projected posteriorly to define a frame centerline 16. FIG. 5 shows the definition of the neutral frame height as the distance between the first fixation element 10 and the second fixation element 20 when the struts 1-6 are neutral. FIG. 6 illustrates an origin 17. The origin 17 is placed at the center of the fractured end of a bone fragment coupled orthogonally to the first fixation element. In a Cartesian coordinate system, the origin 17 is defined as (0,0,0).

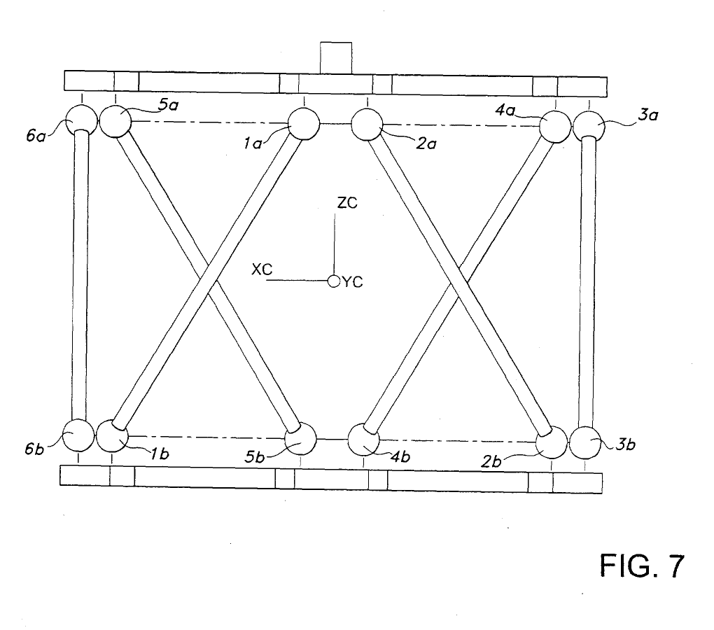

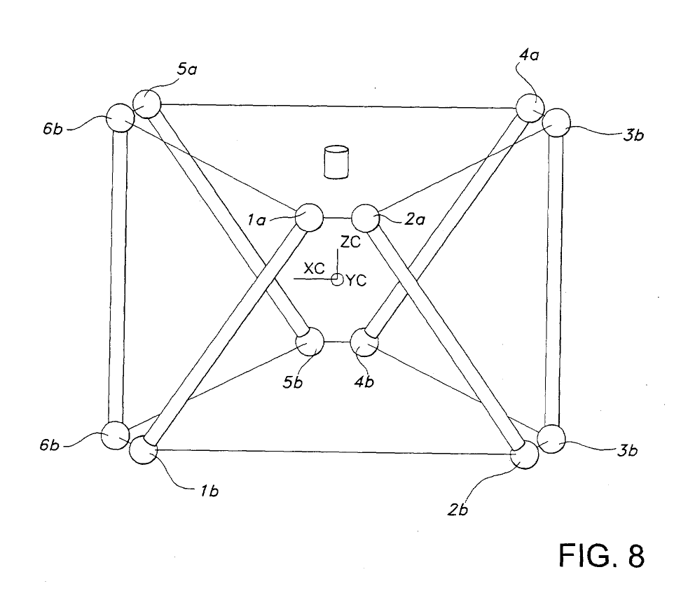

FIGS. 1 and 4 also show U-joints near the end of each strut 1-6. FIG. 1 shows the U-joints as they actually appear near the end of each strut 1-6, and FIG. 4 shows each strut virtually represented with a sphere centered at the respective U-joint's center of rotation. For example, struts 1 and 2 are shown to have proximal U-joints 1a and 2a, and distal U-joints 1b and 2b. The proximal U-joints 1a-6a define a plane A, and the distal U-joints 1b-6b define a plane B as illustrated in FIGS. 7 and 8.

The combination shown in FIG. 3 is operable to bring a first bone fragment into alignment with a second bone fragment. To accomplish an alignment, a user must provide parameters regarding characteristics of the fixation device 100 used. In addition, characteristics of the deformity to be corrected and the way the device 100 is mounted must be input. Given this information from a user, embodiments of the invention provide lengths to which struts can be configured to achieve alignment.

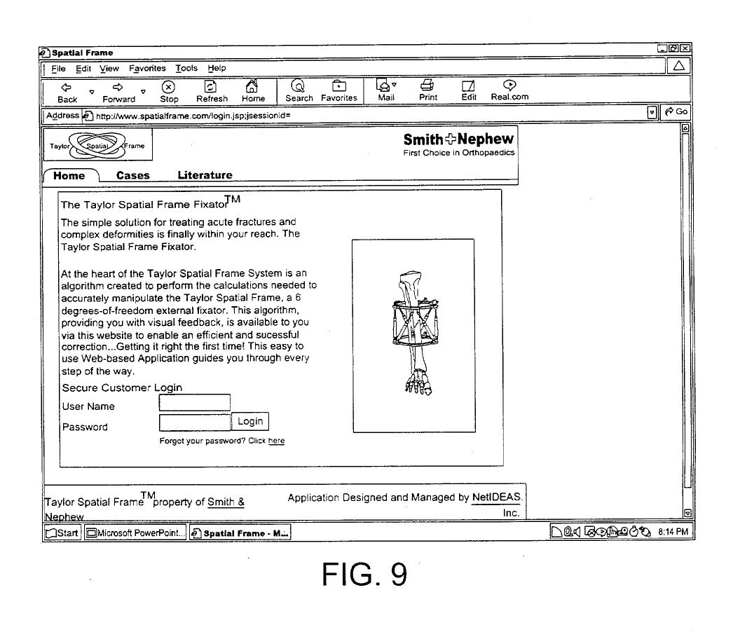

FIGS. 9-21 illustrate an example of an alignment solution reached using a program that receives parameters from a user and outputs strut length settings. FIG. 9 is a depiction of a user login screen designed to provide secure and confidential access to the program.

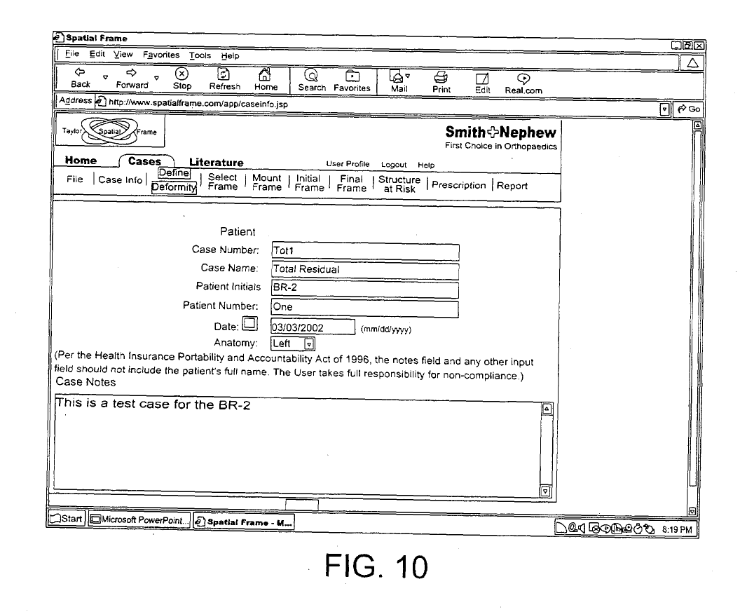

A user completes the fields shown in FIGS. 10-12 to provide information to the program regarding the deformity to be corrected. In FIG. 10 next to “Anatomy,” a user inputs whether a left or right limb is to be corrected. In the example, a left limb is being corrected.

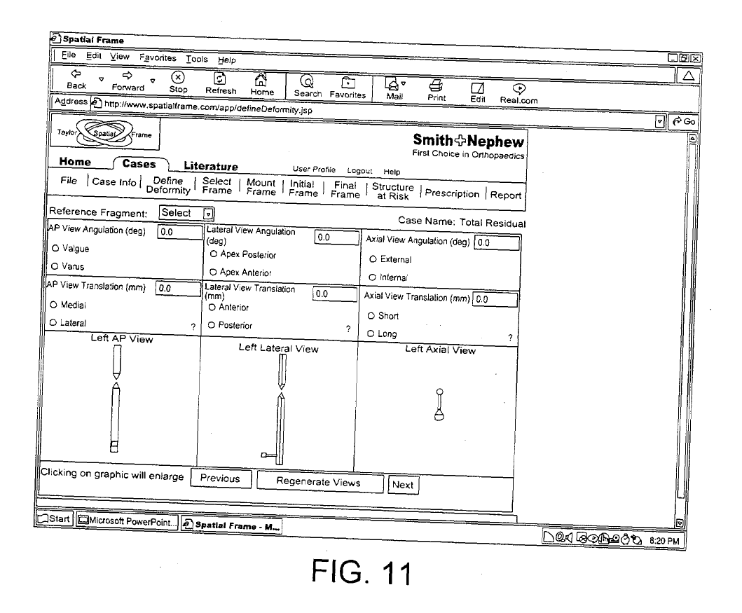

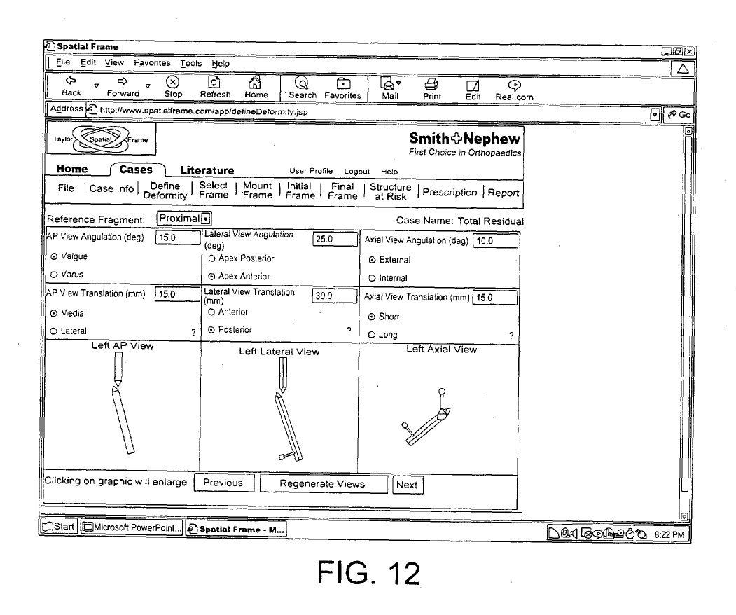

FIGS. 11 and 12 show blanks to be filled in by a user regarding the orientation and extent of a deformity. A selection is required to define which fragment of bone will be a reference fragment, the proximal or the distal. The fragment defined as the reference fragment will be shown as remaining fixed and the other fragment will be brought into alignment with the reference fragment. “Proximal” is selected in the example. The remainder of the parameters to be input are typical clinical parameters that medical professionals are familiar with obtaining. In the example illustrated, the deformity as viewed from the AP is defined by 15.0 degrees of valgus angulation and 15.0 mm of medial translation. As viewed from the lateral, there are 25.0 degrees of apex anterior angulation, and 30.0 mm of anterior translation. Axially, the deformity has 10.0 degrees of external rotation and shows 15.0 mm of shortened axial translation. It is usual to obtain such parameters from x-ray machines and other such imaging devices as well as by observation and physical measurement.

The graphical representations of the present invention labeled “Left AP View”, “Left Lateral View”, and “Left Axial View” are very useful because they provide the user immediate feedback as to whether the correct parameters have been input. The Left AP View and Left Lateral View are particularly familiar and efficient because they correspond to typical x-ray images that the user will likely have available. The embodiment illustrated represents bones as cylindrical objects and a foot on the distal fragment as a perpendicular cylindrical object with a knob at the object's free end. Other embodiments of the invention represent bones with their actual anatomical shapes and proportions. Such representations can be useful to give a user further means of verifying the accuracy of data being input and solutions generated. The use of actual anatomical shapes is carried forward throughout the alignment process in some embodiments. In addition, in some embodiments, soft tissue such as but not limited to muscle, skin, vessels, arteries, and nerves are represented graphically.

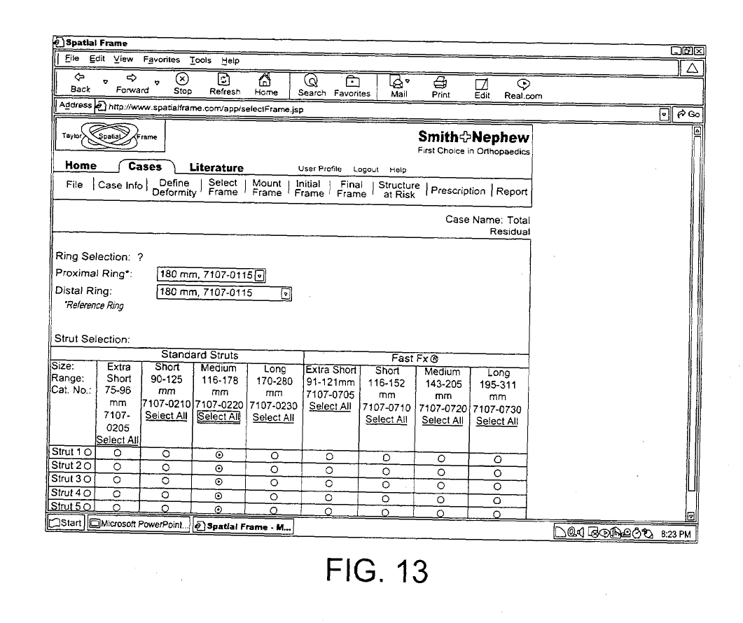

FIG. 13 is an illustration of the input screen used to select what fixation elements, i.e., rings, and what struts will be used in the case. Since rings and struts are stocked items, pull-down menus are provided that only allow a user to select from a limited number of items. This reduces the likelihood of mistakes and increases the accuracy of the alignment. In the example illustrated, both distal and proximal rings are 180 mm rings. The struts selected are standard medium struts, adjustable between 116 mm and 178 mm.

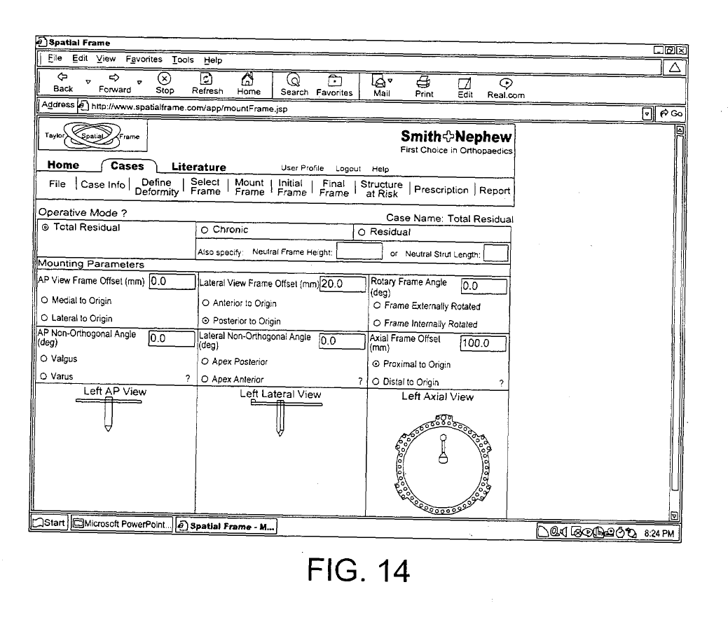

FIG. 14 shows the input screen where a user selects how the first fixation element, or reference ring, of a frame has been or will be mounted on a first bone fragment. This is also where a user defines the operative mode: Total Residual, Chronic, or Residual. As discussed above, residual and chronic solutions generally are known in the art and require going to or coming from a neutral frame. Consequently, for Residual and Chronic modes, either a neutral frame height or neutral strut lengths must be defined. The Total Residual mode is useful in aligning fragments in any circumstance, including the crooked-frame/crooked-bone situation. The origin is defined as the center of the fractured end of the first bone fragment, and the position of the frame is defined relative to that origin. Reference points on the first fixation element of the frame are the center of the fixation element for lateral and AP views, the closest edge of the first fixation element to the origin axially, and the plane defining the master tab to the center of the fixation element for rotary frame angle. Adjustments are also provided to correct for non-orthogonal mountings AP and laterally. In the example shown, there is no AP offset or angulation, no lateral non-orthogonal angulation, and a 20.0 mm posterior to origin frame offset. Axially there is no frame rotary angulation, but there is 100.0 mm proximal to origin axial frame offset. As with the deformity definition, graphical displays of the mounted first fixation element and first bone fragment are provided so that the user may check for proper input of data. The input of data to the stage so far described allows for the relative locations of the first fixation element and the first bone fragment to be stored in the computer.

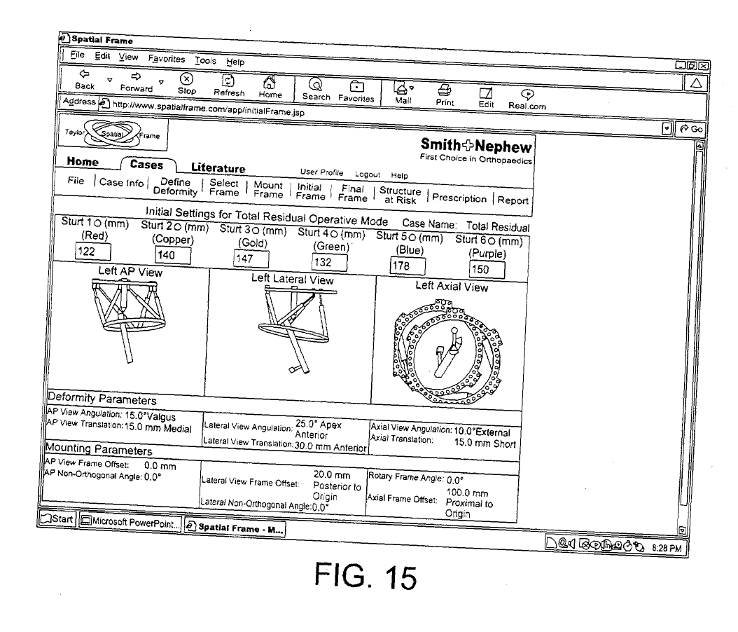

FIG. 15 shows the input screen for the initial frame strut lengths. Typically, these strut lengths are read from the six struts after the second fixation element is coupled to the second bone fragment. In the example that is shown, strut 1 was observed to have a length of 122 mm, strut 2, 140 mm, strut 3, 147 mm, strut 4, 132 mm, strut 5, 178 mm, and strut 6, 150 mm. Once input, the program displays graphic representations of the fixation and bone fragments so that the user may verify the data thus far input into the program.

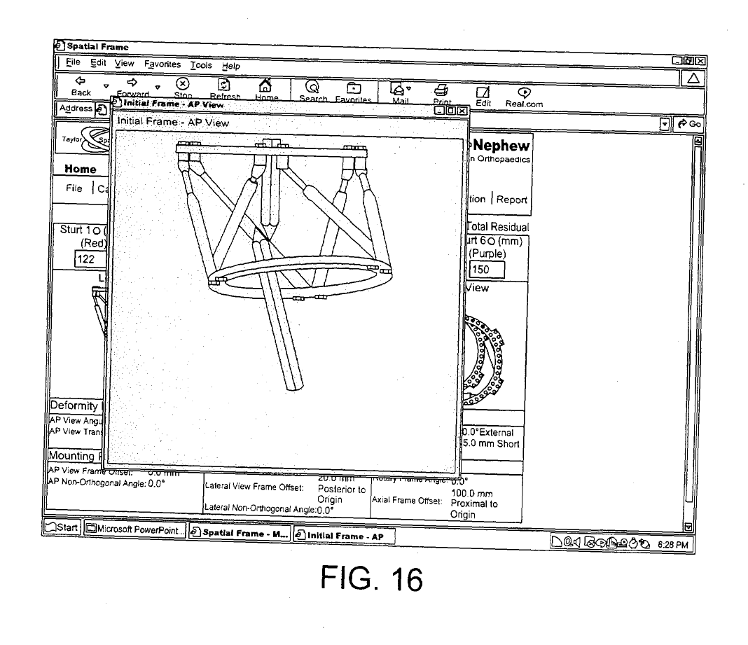

FIG. 16 shows an enlarged view of the Left AP View generated in FIG. 15. In some embodiments of the invention, such enlargements are available for each of the graphic representations of FIG. 15 by selecting the graphic representations. With this data input, the relative locations of the first fixation element and the first bone fragment may be stored in the computer. Additionally, the locations of the couplings of the first and second ends of the first, second, third, fourth, fifth, and sixth adjustable length struts relative to the first and second fixation elements respectively are stored after the orientation of the fixation device is defined by the placement of the second fixation element and the struts. Spatial association between the stored locations of first fixation element and the second fixation element may then be accomplished, thereby storing the locations of the two elements on a common coordinate system.

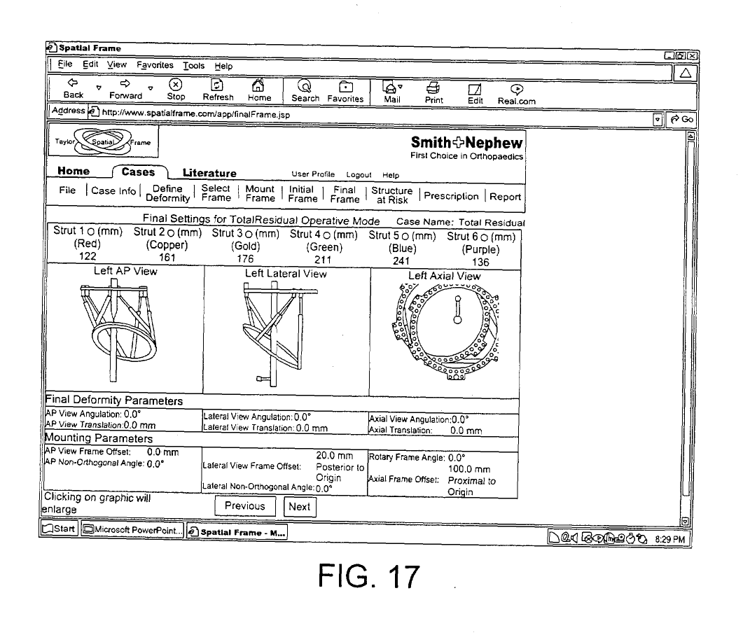

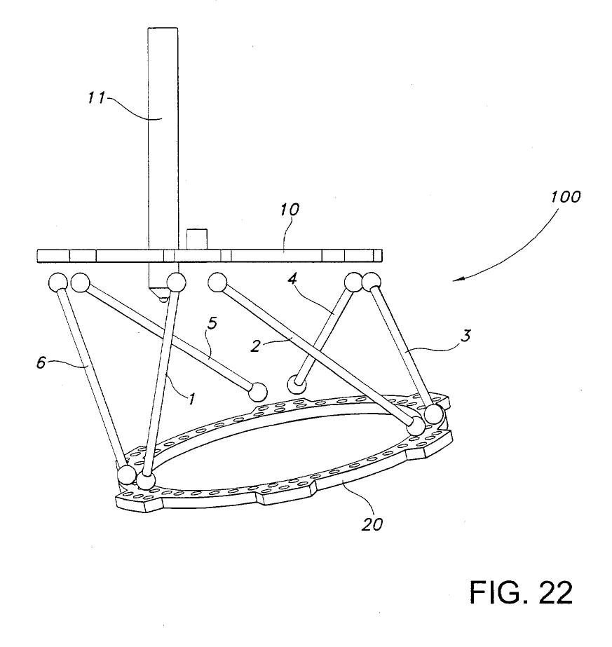

The results of solving for the Final Frame, i.e., spatial association and alignment, are illustrated in FIG. 17. In this example, the resulting strut lengths are: strut 1, 122 mm, strut 2, 161 mm, strut 3, 176 mm, strut 4, 211 mm, strut 5, 241 mm, and strut 6, 136 mm. To reach the results illustrated, embodiments of the invention align the computer generated representations of the stored location of the first bone fragment and a computer generated representation of the stored location of the second bone fragment. Spatial association and aligning may be at least in part enabled by use of a computer assisted engineering program. For example, there exist in the prior art formulas for transforming one fixation element of a Stewart platform relative to another fixation element of the platform to achieve an alignment of bone fragments coupled to each fixation element. Smith & Nephew's U.S. Pat. No. 5,971,984 and numerous Stewart platform manipulation algorithms provide examples of such transformation equations. However, coordinates for both fixation elements must be known to implement the formulas. With a crooked-frame/crooked-bone situation, there previously was no acceptable way to associate the fixation elements and enable the bone fragments to be aligned. A computer assisted engineering program can be used to provide the coordinates of a first fixation element relative to a second fixation element of a Stewart platform where the lengths of the struts are known. Therefore, by modeling a Stewart platform in a computer assisted engineering system, knowledge of the strut lengths is equivalent to knowing the relative coordinates of both fixation elements. Given the coordinates of the first fixation element and the second fixation element and the frame parameters, deformity parameters, and mounting parameters, known transformation equations are used in some embodiments to determine strut lengths required to align the bone fragments. In other embodiments it is possible to achieve alignment by manipulation of graphical representations of the fragments. More specific examples of solving for the Final Frame strut lengths such as those shown in FIG. 17 are provided below in association with FIGS. 22-28.

Recent improvements in computer assisted engineering programs have enabled the programs to simultaneously track both the first and second fixation elements and all six struts. By use of such computer assisted engineering programs, direct use of even the previously applied transformation equations may be bypassed. Consequently, these improved programs have enabled graphical manipulation and measurement of the structures with less user intervention.

Strut lengths may also be solved for using trial and error or a similar iterative method. To implement a trial and error method, start with the assumption that the parameters defining how a bone is mounted on a frame are unchanged and correct. Deformity parameters can be substituted into the known mathematical equations of a residual mode correction, i.e., transformation equations, until the actual crooked-frame strut lengths are achieved. When valid substitutes are found, the actual crooked-frame strut lengths and the deformity parameters of a bone if the bone would be corrected by a residual correction are known. The actual bone would not, however, be corrected by a residual correction because the substituted deformity parameters are not the actual deformity parameters. Another set of x-rays must be taken to determine the actual deformity. The actual deformity parameters observed on the x-rays are then subtracted from the deformity parameters obtained by substitution. The resulting deformity parameters are substituted into the mathematical equations in a residual correction mode, and final strut settings are output. The mathematical equations may be embodied in a computer program.

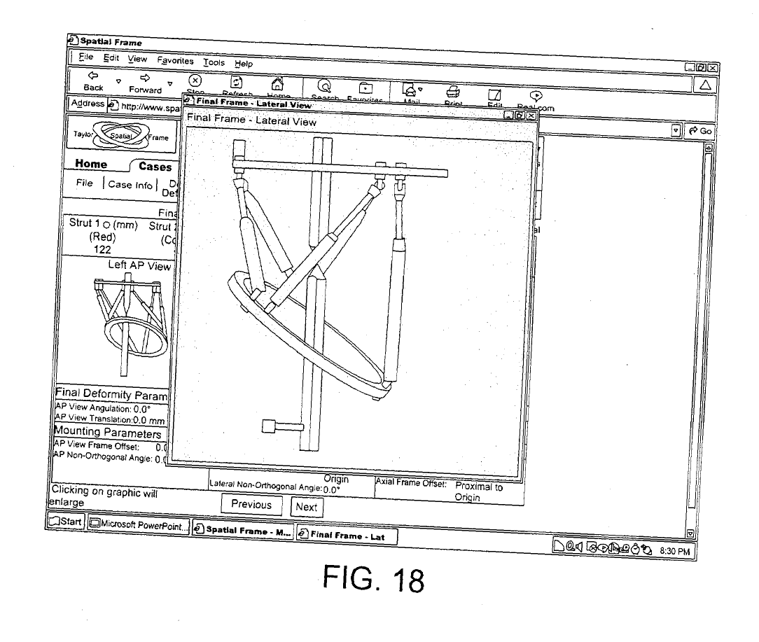

The lengths of the struts when the first and second fragments are aligned are provided in the output of FIG. 17. In addition, graphical representations of the solution are provided so that the user may check the progress and accuracy of the alignment. FIG. 18 shows an enlarged view of the “Left Lateral View” generated in FIG. 17. In some embodiments of the invention, such enlargements are available for each of the graphic representations of FIG. 17 by selecting the graphic representations.

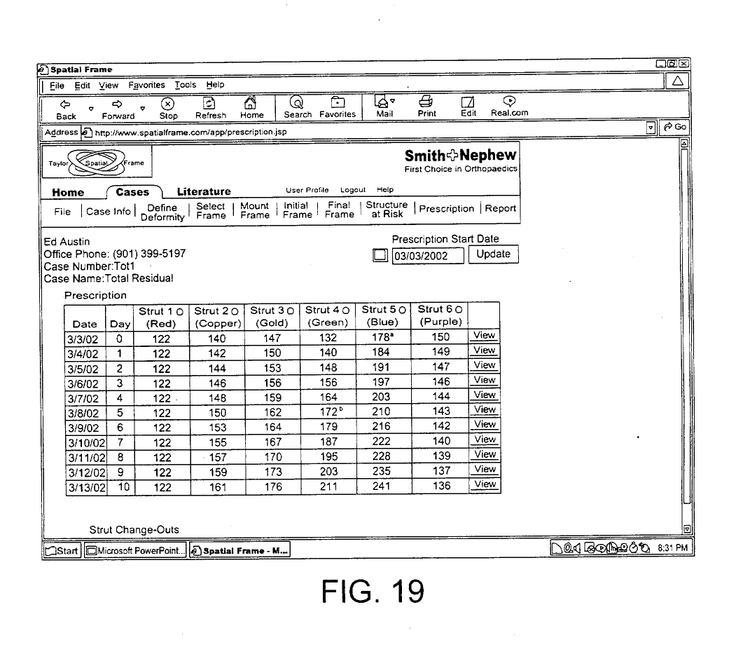

FIGS. 19-21 illustrate a prescription for aligning the fragments over a ten-day period. The rate of alignment can be metered to not exceed a certain amount of distance moved in a given time or can be set to achieve completion in a given amount of time. A factor that is often important in determining a rate of alignment is whether there may be “structures at risk” during the alignment such as nerves, vessels, muscle, skin, arteries, or other tissue. A structure at risk is tissue that may be damaged by too rapid of an alignment. Therefore, the rate of alignment is controlled in some circumstances.

Embodiments of the invention not only allow for protection of structures at risk by controlling the rate at which alignments are made, but also enable the control of the path taken to achieve an alignment. A path may be chosen that minimizes stress on a structure at risk. Alternatively, a user can specify a path for bone fragments to travel that causes a fractured end of the first bone fragment to avoid contact with a fractured end of second bone fragment until immediately prior to completion of the alignment. The term “immediately prior” means within a later portion of the time period of the correction. For example, the bone fragments could be scheduled for a path that would prevent their ends from contacting one another and potentially creating further damage to the ends. However, near the completion of the alignment, the bone fragments would need to be brought into contact for proper healing of the bone. In other embodiments, the bone ends could be initially brought together and rotated into place while maintaining contact throughout the alignment.

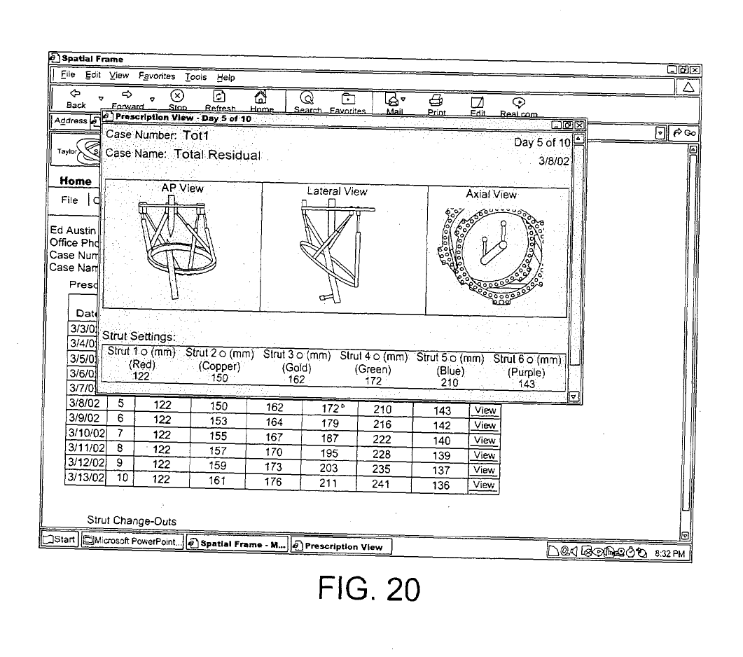

FIG. 20 shows an example of an enlarged view that graphically represents the progress of an alignment. Such views are available for each day of the alignment by selecting the “View” column to the far right of the prescription shown (FIG. 19). This feature is useful for checking progress and accuracy.

In some embodiments of the invention, frame configurations such as those shown in FIGS. 15 and 17, and the progress representations available by selecting “View” are useful in determining whether coupling structures such as pins and wires are likely to interfere with struts and fixation elements during the course of an alignment. A visual inspection of the representations is useful to determine interference in some circumstances. Additionally, the pins and wires themselves may be modeled and tracked in some embodiments of the invention.

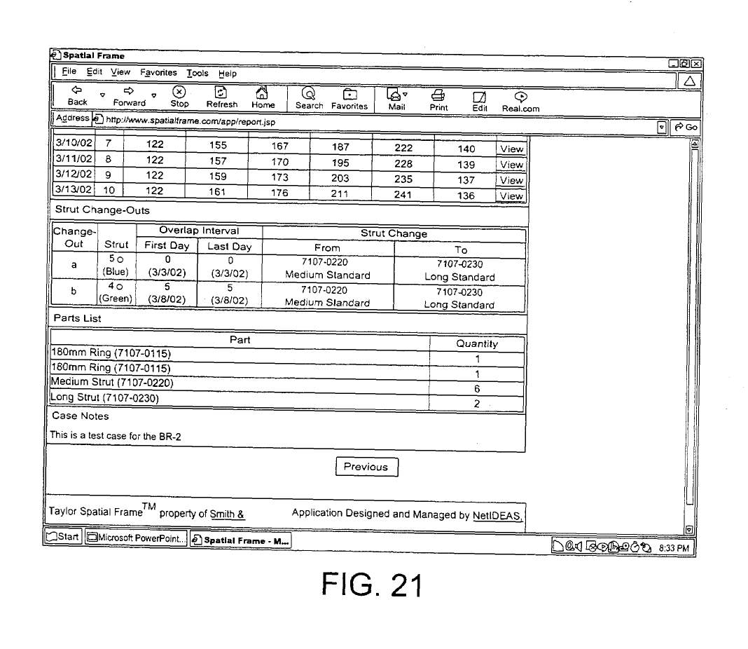

Footnotes “a” and “b” (FIGS. 19 and 21) designate when struts must be changed due to struts needing to lengthen or shorten beyond the physical limits of a strut. In some embodiments of the invention, the configuration of the fixation device and selection of struts is optimized by the program itself. For example, during the process of preoperative or intraoperative planing, if a proposed alignment was determined to result in exceeding a strut parameter before alignment would be achieved, placement of the second fixation element could be altered to avoid strut replacement. Such an embodiment avoids the additional cost of replacement struts.

FIGS. 22-28 illustrate aspects of methods of configuring an orthopaedic fixation device that can be coupled to fragments of a fractured bone. Such methods are useful in determining the adjustment required to align the fragments. FIGS. 22-25 and 26-28 respectively illustrate two ways of accomplishing embodiments of the invention. As described above, a user can input frame parameters, mounting parameters, and strut settings to virtually represent the fixation device and the fragments in three-dimensional space. In some embodiments of the invention, the representations of the fixation device and the fragments are accomplished by storing data in a computer.

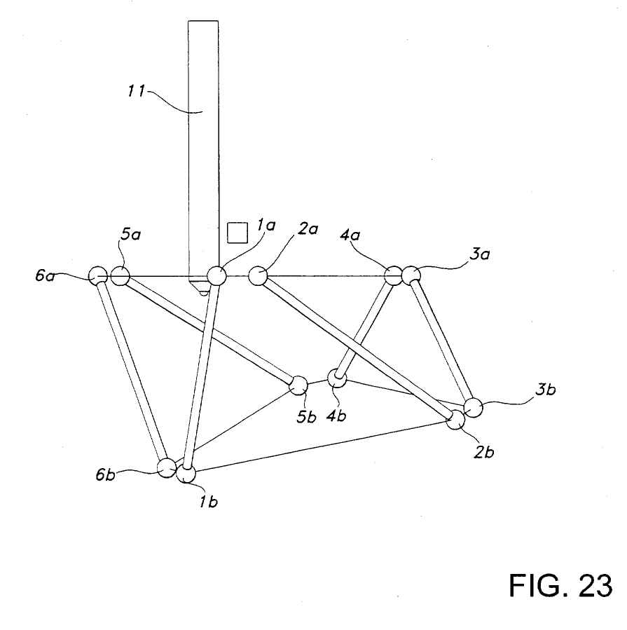

With information regarding the representations of the fixation elements and the bone fragments known (e.g., frame parameters, mounting parameters, and strut settings), spatial associations among the representations of the fixation elements and bone fragments are determinable. Such a determination can be made numerically by use of a Cartesian coordinate system and the geometries of the fixation device components, or by representing the elements graphically, such as in a computer assisted engineering program. FIG. 22 shows representations of a first fixation element 10, a second fixation element 20 and a first bone fragment 11 represented in three-dimensional space and spatially associated with one another. As illustrated in FIG. 23, this embodiment of the invention further relates representations of the first fixation element 10 with proximal U-joints 1a-6a and the second fixation element 20 with distal U-joints 1b-6b. The first fixation element 10 and the proximal U-joints 1a-6a Cartesian coordinates are therefore determinable from the mounting parameters. Then, knowing the strut settings and, either by use of transformation equations or modeling in a computer assisted engineering program, the Cartesian coordinates of the distal U-joints 1b-6b are determinable. Because there is a constant and predetermined spatial relationship between the first fixation elements and their respective U-joints, tracking the positions of the U-joints is equivalent to tracking the positions of the fixation elements.

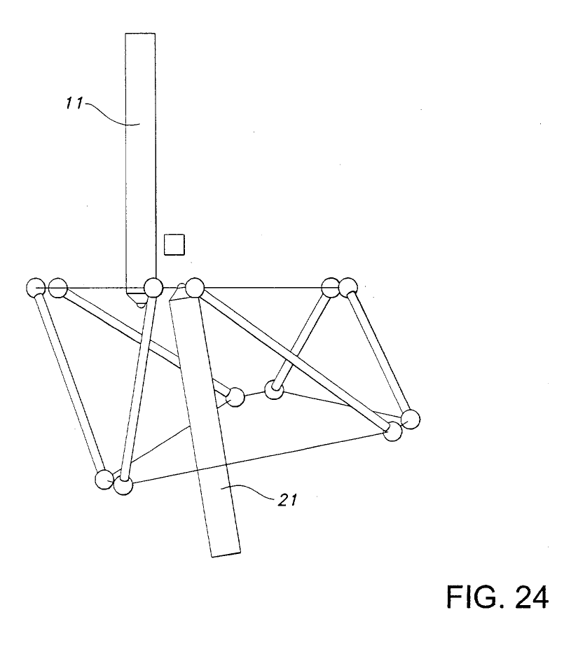

FIG. 24 depicts a representation of a second bone fragment 21 that is spatially associated with the other represented elements of the external fixation device, including the second fixation element 20 (FIG. 22). The input deformity parameters enable the association of the second bone fragment 21. Spatial association is also made between the representations of the first bone fragment 11 and second bone fragment 21.

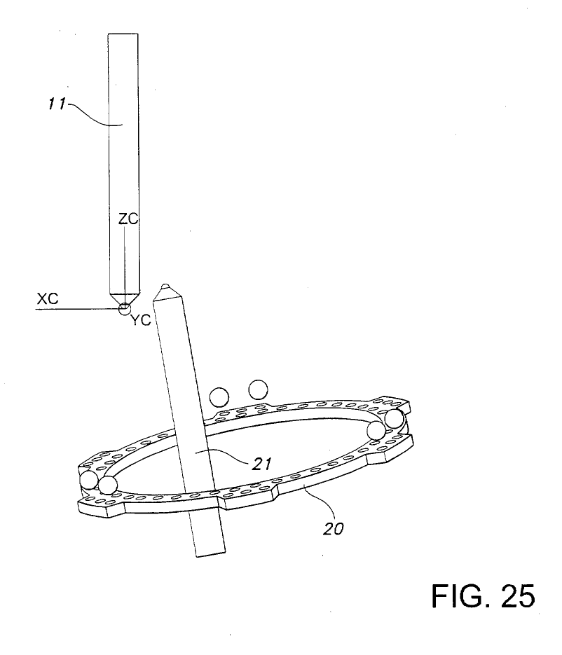

FIG. 25 shows the representation of the second fixation element 20 spatially associated with the representation of the second bone fragment 21. By transforming the representation of the second bone fragment 21 to align with the representation of the first bone fragment 11, and tracking the representation of the second fixation element 20 as it acts with the representation of the second bone fragment 21, new coordinates for the second fixation element 20 can be determined.

Because the spatial associations of the representations of the first and second fixation elements 10 and 20 are known in the embodiment of the invention illustrated, Cartesian coordinates can be derived for the fixation elements, and the associated U-joints. In some embodiments of the invention, a computer assisted engineering program is used to determine these coordinates. The coordinates may be used in conjunction with data about the deformity of the bone and known transformation equations to determine the amount that the struts 1-6 must be adjusted to align the bone fragments. The transformation equations in effect track the spatially associated locations of the representations of the first fixation element 10 and the second fixation element 20 to provide strut lengths that will generate the alignment of the bone fragments.

The alignment of the virtual representations of the first bone fragment 11 and the second bone fragment 21 may also be accomplished by aligning virtual representations of the bone fragments, such as by manipulating images depicted by a computer assisted engineering program.

In a further example, consider the first bone fragment 11 as sitting along a line defined by points at the proximal and distal ends of the first bone fragment 11. The first fixation element 10 is spatially associated in relation to the line along which the first bone fragment 11 sits. Likewise, the second bone fragment 21 may be defined as sitting along a line defined by points at the fragment's proximal and distal ends. The second fixation element 20 is spatially associated in relation to the line along which the second bone fragment 21 sits. A computer assisted engineering program may be used to establish the relative positions of the first fixation element 10 and the second fixation element 20, given the strut lengths between the fixation elements. To align representations of the first bone fragment 111 and the second bone fragment 21, the proximal end of the second bone fragment 21 is virtually moved to be coincident with the distal end of the first bone fragment 11. The distal end of the second bone fragment 21 may then be rotated about the proximal end of the second bone fragment 21 until the distal end is located on the line defined by the first bone fragment 11. The distance, direction, and rotation of the transformation required to move the second bone fragment 21 are applied to the second fixation element 20. Transformations of this type can be accomplished mathematically or by manipulating images displayed through a computer assisted engineering program. Note that in the art known prior to the present invention, these transformations were not possible with respect to the fixation elements and struts because the location of the second fixation element 20 relative to the first fixation element 10 was not determinable under the equations then applied, unless the frame was a neutral frame. With the transformed second fixation element 20 position known relative to the first fixation element 10, the lengths of the struts are readily determinable mathematically or graphically.

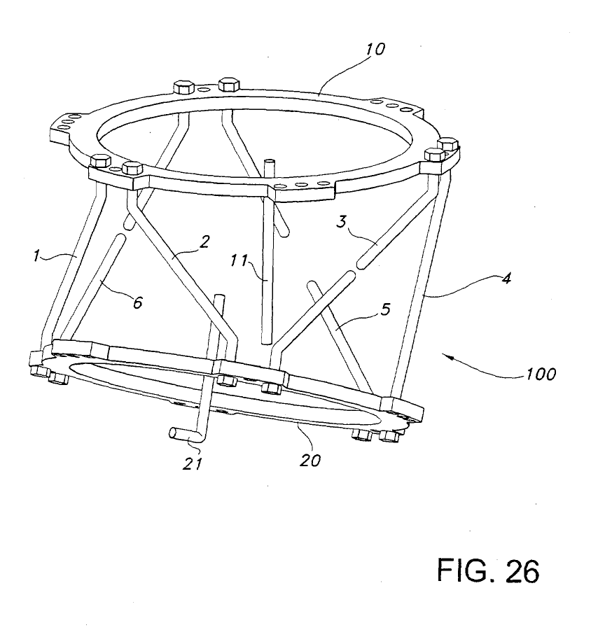

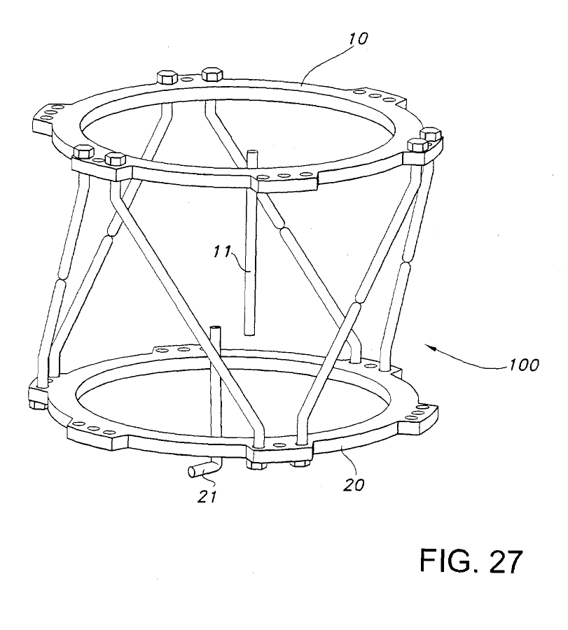

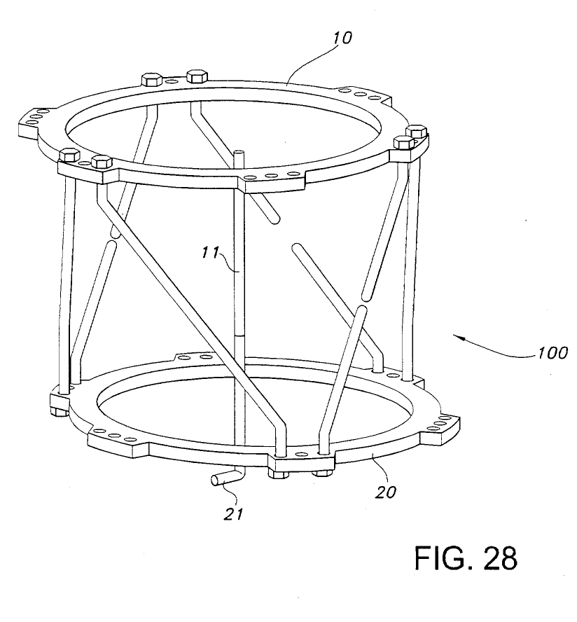

FIGS. 26-28 illustrate an alternate way of accomplishing an alignment under embodiments of the invention. FIG. 26 shows representations of the first fixation element 10, the second fixation element 20, the first bone fragment 11, and second bone fragment 21. As in previous embodiments, the fixation elements and bone fragments are virtually represented or modeled and associated to one another based on the frame parameters, mounting parameters, and strut settings provided by a user. However, an alternate method of alignment is shown in FIGS. 27 and 28. With all of the elements and fragments modeled, the fixation device 100 can be virtually returned to any neutral frame (FIG. 27). The fixation elements and the bone fragments will continue to be tracked virtually. Virtual deformity parameters are then observed. Typical clinical views such as the AP, lateral, and axial views, like those shown in FIG. 15, are observed in the virtual bone fragments to determine the virtual deformity parameters. The virtual deformity parameters are then used in known transformation equations to determine strut lengths for an alignment as is shown in FIG. 28. Stated another way, once the virtual deformity parameters are determined (FIG. 27), a “Residual” rather than a “Total Residual” may be run to determine final strut settings needed for an alignment of the bone fragments.

In embodiments of the invention, a path for the fragments to travel may be specified so that desirable modes of alignment can be achieved as discussed above.

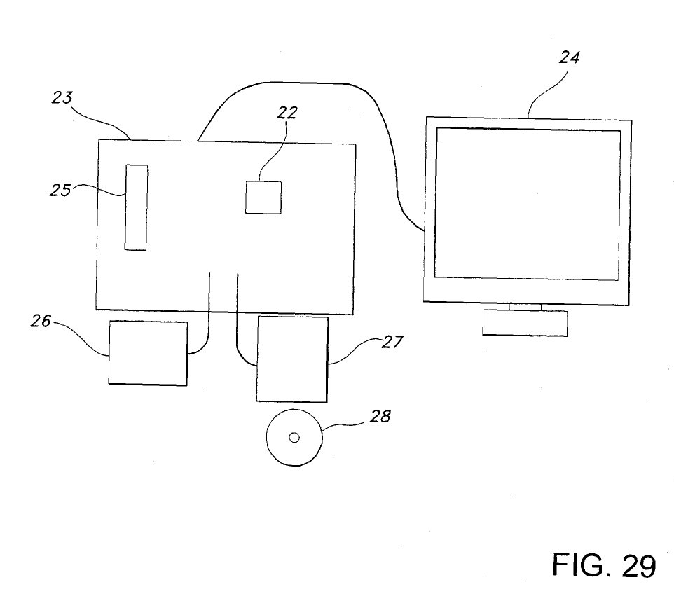

While the embodiments of the invention that have been specifically detailed here include six strut ring external fixation structures, it is important to note that the apparatuses and methods of the invention are applicable to many types of external fixation devices. Many variations of the Smith & Nephew, Inc. Stewart platform based external fixators are noted in the patents and documents incorporated by reference above. Apparatuses and methods of the invention are useful with any of these variations, including with external fixators that have only partial rings, reduced numbers of struts, or include clamp and bar structures built into or built separately from the external fixation device. Apparatuses and methods of the invention are equally useful in configuring unilateral orthopaedic external fixation devices. Varieties of such unilateral devices are illustrated in FIGS. 28 and 29 of U.S. Pat. No. 5,702,389. The illustrated devices also incorporate a six strut Stewart platform. However, a unilateral orthopaedic external fixation device within the claims of this invention would not necessarily include a Stewart platform. A device with the claims of this invention may merely include a combination of adjustments that allow the device to mimic some or all of the degrees of translation and rotation of the devices detailed above.

FIG. 29 illustrates a digital computing device programmed to provide data to a user for adjusting an orthopaedic fixation device 100. A central processing unit 22 is shown electrically coupled to a motherboard 23. The central processing unit 22 is for executing program instructions. A monitor 24 is also electrically coupled to the motherboard 23. The monitor 24 is for displaying representations of the fixation device 100. A random access memory device 25 is electrically coupled to the motherboard 23. A hard disk drive 26 is electrically coupled to the motherboard 23. A removable media disk drive 27 is electrically coupled to the motherboard 23. Each of the random access memory device 25, the hard disk drive 26, and the removable media disk drive 27 are capable of storing program instructions that enable actions to adjust the orthopaedic fixation device. In some embodiments of the invention, two or more of the central processing unit 22, the motherboard 23, the monitor 24, the random access memory device 25, the hard disk drive 26, and the removable media disk drive 27 may be integrated into a single component. Such components may be referred to as a system-on-a-chip.

The instructions executed by the digital computing device of FIG. 29 are consistent with the apparatus and method embodiments described above. The digital computing device may be a single computer system such as computer system 201 illustrated in FIG. 3. Alternatively, the digital computing device may be two or more computer systems, such as computer systems 201 and 202 connected through a network 203.

Another embodiment of the invention is a program storage device 28 (FIG. 29) containing instructions that enable a computer to provide data specifying how to configure an orthopaedic fixation device that can be coupled to fragments of a fractured bone. The instructions stored on the program storage device 28 are consistent with the apparatus and method embodiments described above.

Another use for an embodiment of the device is joint contracture or other such exercise or articulation of a joint. In an instance where there has been trauma, atrophy, or some other abnormality experienced by a patient near a joint, soft tissue may become damaged. Soft tissue damage may include damage to muscles, skin, tendons, ligaments, cartilage, etc. A result of damage is sometimes an inability to fully flex or extend a joint. An embodiment of the invention is useful to couple fixation elements to bones on either side of the joint and use the fixation device to flex and/or extend the limb about the joint. Just as with bone alignment, a prescription can be created to reposition the fixation elements relative to one another. In embodiments for causing movement about a joint, the natural center of the joint would typically be set as a rotation point about which the fixation device would operate.

What is claimed is:

1. An external orthopaedic fixation device in combination with a computer, the combination for aligning fragments of a fractured bone comprising:
a first fixation element for coupling to a first bone fragment;
a second fixation element for coupling to a second bone fragment;
a first adjustable length strut coupled at its first end to the first fixation element and coupled at its second end to the second fixation element;
a second adjustable length strut coupled at its first end to the first fixation element and coupled at its second end to the second fixation element;
a third adjustable length strut coupled at its first end to the first fixation element and coupled at its second end to the second fixation element;
a fourth adjustable length strut coupled at its first end to the first fixation element and coupled at its second end to the second fixation element;
a fifth adjustable length strut coupled at its first end to the first fixation element and coupled at its second end to the second fixation element; and
a sixth adjustable length strut coupled at its first end to the first fixation element and coupled at its second end to the second fixation element;
wherein when the first bone fragment and the second bone fragment are out of alignment, at least two of the first, second, third, fourth, fifth, and sixth adjustable length struts are different lengths, and if the first, second, third, fourth, fifth, and sixth adjustable length struts were any same length, the first bon e fragment and th e second bone fragment would be out of alignment;
wherein the combination is operable to bring the first bone fragment into alignment with the second bone fragment by:
storing the relative locations of the first fixation element and the first bone fragment,
storing the locations of the couplings of the first ends of the first, second, third, fourth, fifth, and sixth adjustable length struts relative to the first fixation element,
storing the relative locations of the second fixation element and the second bone fragment,
storing the locations of the couplings of the second ends of the first, second, third, fourth, fifth, and sixth adjustable length struts relative to the second fixation element,
spatially associating the stored location of the first fixation element with the stored location of the second fixation element,
aligning a computer generated representation of the stored location of the first bone fragment relative to a computer generated representation of the stored location of the second bone fragment,
obtaining the respective distances in the aligned computer generated representations between the first and second ends of the first, second, third, fourth, fifth, and sixth adjustable length struts respectively, and
providing the aligned lengths of the first, second, third, fourth, fifth, and sixth adjustable length struts to a user for adjusting the adjustable length struts of the external orthopaedic fixation device;
wherein the computer includes two or more computers linked together over a network.

2. The combination of claim 1 wherein the network is the Internet.

3. An external orthopaedic fixation device in combination with a computer, the combination for aligning fragments of a fractured bone comprising:
a first fixation element for coupling to a first bone fragment;
a second fixation element for coupling to a second bone fragment;
a first adjustable length strut coupled at its first end to the first fixation element and coupled at its second end to the second fixation element;
a second adjustable length strut coupled at its first end to the first fixation element and coupled at its second end to the second fixation element;
a third adjustable length strut coupled at its first end to the first fixation element and coupled at its second end to the second fixation element;
a fourth adjustable length strut coupled at its first end to the first fixation element and coupled at its second end to the second fixation element;
a fifth adjustable length strut coupled at its first end to the first fixation element and coupled at its second end to the second fixation element; and
a sixth adjustable length strut coupled at its first end to the first fixation element and coupled at its second end to the second fixation element;
wherein when the first bone fragment and the second bone fragment are out of alignment, at least two of the first, second, third, fourth, fifth, and sixth adjustable length struts are different lengths, and if the first, second, third, fourth, fifth, and sixth adjustable length struts were any same length, the first bone fragment and the second bone fragment would be out of alignment;
wherein the combination is operable to bring the first bone fragment into alignment with the second bone fragment by:
storing the relative locations of the first fixation element and the first bone fragment,
storing the locations of the couplings of the first ends of the first, second, third, fourth, fifth, and sixth adjustable length struts relative to the first fixation element,
storing the relative locations of the second fixation element and the second bone fragment,
storing the locations of the couplings of the second ends of the first, second, third, fourth, fifth, and sixth adjustable length struts relative to the second fixation element,
representing the first, second, third, fourth, fifth, and sixth adjustable length struts, the first fixation element, and the second fixation element in a computer assisted engineering program such that the relative locations of the first fixation element and the second fixation element are determinable while simultaneously altering the represented lengths of the first, second, third, fourth, fifth, and sixth struts in the computer assisted engineering program,
aligning a computer generated representation of the stored location of the first bone fragment relative to a computer generated representation of the stored location of the second bone fragment,
obtaining the respective distances in the aligned computer generated representations between the first and second ends of the first, second, third, fourth, fifth, and sixth adjustable length struts respectively, and
providing the aligned lengths of the first, second, third, fourth, fifth, and sixth adjustable length struts to a user for adjusting the adjustable length struts of the external orthopaedic fixation device.

4. The combination of claim 3 wherein the computer includes two or more computers linked together over a network.

5. The combination of claim 4 wherein the network is the Internet.

6. A method of configuring an orthopaedic fixation device that can be coupled to fragments of a fractured bone comprising the acts of:
representing a first fixation element of the fixation device virtually in three-dimensional space;
representing a first bone fragment virtually in three-dimensional space;
spatially associating the representation of the first fixation element with the representation of the first bone fragment;
representing a second fixation element of the fixation device virtually in three-dimensional space;
representing a second bone fragment virtually in three-dimensional space;
spatially associating the representation of the second fixation element with the representation of the second bone fragment;
representing the first fixation element and the second fixation element in a computer assisted engineering program such that the computer assisted engineering program dynamically tracks the first fixation element and the second fixation element;
spatially associating the representation of the first bone fragment with the representation of the second bone fragment;
aligning the virtual representation of the first bone fragment with the virtual representation of the second bone fragment while tracking the spatially associated locations of the representation of first fixation element and the representation of the second fixation element; and
configuring the orthopaedic fixation device such that the first fixation element is in the same relative position to the second fixation element as the aligned representation of the first fixation element is with the aligned representation of the second fixation element.

7. A digital computing device programmed to provide data to a user for adjusting an orthopaedic fixation device that can be coupled to fragments of a fractured bone comprising:
a motherboard;
a central processing unit electrically coupled to the motherboard for executing program instructions;
a monitor electrically coupled to the motherboard for displaying representations of the fixation device; and
a memory device electrically coupled to the motherboard that stores program instructions that enable the computing device to:
represent a first fixation element of the fixation device virtually in three-dimensional space;
represent a first bone fragment virtually in three-dimensional space;
spatially associate the virtual representation of the first fixation element with the virtual representation of the first bone fragment;
represent a second fixation element of the fixation device virtually in three-dimensional space;
represent a second bone fragment virtually in three-dimensional space;
spatially associate the virtual representation of the second fixation element with the virtual representation of the second bone fragment;
spatially associate the virtual representation of the first bone fragment with the virtual representation of the second bone fragment;
align the virtual representation of the first bone fragment with the virtual representation of the second bone fragment while tracking the spatially associated locations of the virtual representation of first fixation element and the virtual representation of the second fixation element; and
output data specifying how the first fixation element is to be positioned relative to the second fixation element to align the first bone fragment and the second bone fragment;
wherein the computing device includes two or more computers linked together over a network.

8. The digital computing device of claim 7 wherein the network is the Internet.

9. The digital computing device of claim 7 wherein the memory device is a random access memory device.

10. The digital computing device of claim 7 wherein the memory device is a non-volatile memory device.

11. The digital computing device of claim 7 wherein the program instructions enabling the virtual representation of the first fixation element include computer assisted engineering program instructions.

12. The digital computing device of claim 7 wherein the program instructions enabling the virtual representation of the first bone fragment include computer assisted engineering program instructions.

13. The digital computing device of claim 7 wherein the program instructions enabling the virtual representation of the second fixation element include computer assisted engineering program instructions.

14. The digital computing device of claim 7 wherein the program instructions enabling the virtual representation of the second bone fragment include computer assisted engineering program instructions.

15. The digital computing device of claim 7 wherein the program instructions enabling the aligning of the virtual representations of the bone fragments while tracking virtual representations of the fixation elements are at least in part computer assisted engineering program instructions.

16. The digital computing device of claim 7 wherein the program instructions enabling the aligning of the virtual representations of the bone fragments while tracking virtual representations of the fixation elements include instructions specifying a path for the fragments to travel.

17. The digital computing device of claim 16 wherein the program instructions specifying a path for the fragments to travel specify a path that causes a fractured end of the first bone fragment to avoid contact with a fractured end of second bone fragment until immediately prior to completion of the alignment.
